(19) United States
(12) Patent Application Publication
(10) Pub. No.: US 2026/0000707 A1
(43) Pub. Date: Jan. 1, 2026

(54) Tablet with improved stability

(71) Applicant: NOBELPHARMA CO., LTD., Tokyo (JP)

(72) Inventors: Tatsuo NOMURA, Tokyo (JP); Tsubasa KUBO, Matsudo (JP); Toru YASUZAWA, Hadano (JP)

(73) Assignee: NOBELPHARMA CO., LTD., Tokyo (JP)

(21) Appl. No.: 19/256,227

(22) Filed: Jul. 1, 2025

(30) Foreign Application Priority Data
Jul. 1, 2024 (JP) 2024-106446
Dec. 20, 2024 (JP) 2024-225078

(51) Int. Cl. A61K 33/30; A61K 9/20

(52) U.S. Cl. CPC A61K 33/30; A61K 9/2018; A61K 9/2054; A61K 9/2059; A61K 9/2095

(57) ABSTRACT

A tablet comprising zinc histidine or a solvate thereof as an active ingredient, further comprising one or more ingredients selected from the group consisting of D-mannitol, sucrose, sorbitol, xylitol, corn starch, and potato starch; and crystalline cellulose; and excluding lactose, fructose, and glucose, and a tablet comprising zinc histidine or a solvate thereof as an active ingredient, having a 15-minute dissolution value of 80% or more in the paddle method (test solution: water, rotate speed: 75 rpm) (excluding lactose, fructose, and glucose), obtained by the following steps: granulating zinc histidine or a solvate thereof, one or more components selected from the group consisting of D-mannitol, sucrose, sorbitol, xylitol, corn starch, and potato starch, and crystalline cellulose, with a binder; drying the obtained granulate; and tableting the dried granulate.

### TECHNICAL FIELD

The present invention relates to a tablet containing zinc histidine or a solvate thereof as an active ingredient and having improved stability. More specifically, the present invention relates to A tablet comprising zinc histidine or a solvate thereof as an active ingredient, further comprising one or more ingredients selected from the group consisting of D-mannitol, sucrose, sorbitol, xylitol, corn starch, and potato starch; and crystalline cellulose; and excluding lactose, fructose, and glucose. The present invention also relates to a tablet comprising zinc histidine or a solvate thereof as an active ingredient, and suppressing a decrease in dissolution rate and discoloration of the tablet.

### BACKGROUND ART

Zinc plays various roles depending on different pathological conditions and states, and is easily consumed accordingly, often leading to deficiency or hypozincemia. As a treatment for zinc deficiency, formulations containing zinc histidine are known, and in Germany and South Korea, hard capsules containing zinc histidine are commercially available. Additionally, for example, in Non-Patent Document 1, tablets of zinc histidine prepared using lactose as a binder are disclosed.

### PRIOR ART REFERENCES

### Non-Patent Documents

Non-Patent document 1: Jurgen Scholmerich et al., Am. J. Clin. Nutr.,1987,45,p.1480-6

### SUMMARY OF INVENTION

### Problems to be Resolved by the Invention

Capsules containing zinc histidine that have been commercially available in Germany and other countries use large size 0 capsules. However, considering ease of administration, it is preferable to use a more compact tablet form.

As a tablet containing zinc histidine, Non-Patent Document 1 discloses a preparation containing lactose as an excipient. However, the inventors have shown that using lactose as an excipient causes a decrease in solubility and discoloration during storage.

The present invention aims to provide tablets containing zinc histidine or a solvate thereof as an active ingredient and have sufficient stability. The present invention also aims to provide tablets containing zinc histidine or a solvate thereof as an active ingredient and having reduced dissolution and/or discoloration of the formulation.

The inventors have discovered that the above-mentioned problems can be solved by tablets containing one or more components selected from the group consisting of D-mannitol, sucrose, sorbitol, xylitol, corn starch, and potato starch; and crystalline cellulose; and not containing lactose, fructose, or glucose, and have completed the present invention.

### Means of Solving the Problems

The present invention provides the following embodiments.
Embodiment 1:

A tablet comprising zinc histidine or a solvate thereof as an active ingredient, further comprising one or more ingredients selected from the group consisting of D-mannitol, sucrose, sorbitol, xylitol, corn starch, and potato starch; and crystalline cellulose; and excluding lactose, fructose, and glucose.
Embodiment 2:

The tablet according to embodiment 1, wherein the content of the active ingredient is 40% by mass to 75% by mass of the entire tablet.
Embodiment 3:

The tablet according to embodiment 1, wherein the content of one or more components selected from the group consisting of D-mannitol, sucrose, sorbitol, xylitol, corn starch, and potato starch is 5% by mass to 15% by mass of the entire tablet.
Embodiment 4:

The tablet according to any one of embodiments 1 to 3, wherein the content of crystalline cellulose is 10% by mass to 30% by mass of the entire tablet.
Embodiment 5:

The tablet according to any one of embodiments 1 to 3, wherein the active ingredient is contained in an amount equivalent to 25 mg or 50 mg of zinc.
Embodiment 6:

The tablet according to any one of embodiments 1 to 3, wherein the solvate is a hydrate.
Embodiment 7:

The tablet according to any one of embodiments 1 to 3, comprising zinc histidine hydrate as the active ingredient.
Embodiment 8:

The tablet according to any one of embodiments 1 to 3, wherein the 15-minute dissolution value in the paddle method (test solution: water, rotate speed: 75 rpm) is 80% or more.
Embodiment 9:

The tablet according to embodiment 8, wherein the 15-minute dissolution value in the paddle method (test solution: water, rotate speed: 75 rpm) is 80% or more both at the start of storage and after 3 months under storage conditions of 40° C., 75% RH, and in an open system.
Embodiment 10:

The tablet according to embodiment 9, wherein the 15-minute dissolution value in the paddle method (test solution: water, rotate speed: 75 rpm) decreases by less than 10% from the start of storage to three months under conditions of 40° C., 75% RH, and in an open system.
Embodiment 11:

A tablet comprising zinc histidine or a solvate thereof as an active ingredient, having a 15-minute dissolution value of 80% or more in the paddle method (test solution: water, rotate speed: 75 rpm) (excluding lactose, fructose, and glucose), obtained by the following steps:

granulating zinc histidine or a solvate thereof, one or more components selected from the group consisting of D-mannitol, sucrose, sorbitol, xylitol, corn starch, and potato starch, and crystalline cellulose, with a binder;

drying the obtained granulate; and

tableting the dried granulate.
Embodiment 12:

The tablet according to embodiment 11, wherein the 15-minute dissolution value in the paddle method (test solution: water, rotate speed: 75 rpm) is 80% or more at the start of storage and after 3 months of storage at 40° C., 75% RH, and in an open system.
Embodiment 13:

The tablet according to embodiment 12, wherein the 15-minute dissolution value in the paddle method (test solution: water, rotate speed: 75 rpm) decreases by less than 10% from the start of storage to three months under conditions of 40° C., 75% RH, and in an open system.

### Effects of the Invention

The tablets of the present invention contain zinc histidine or a solvate thereof as an active ingredient. The solvate is not particularly limited as long as it is a pharmaceutically acceptable one. Examples of such solvates include hydrates, organic solvates, and mixed solvates of water and organic solvents. Preferred solvates may include a hydrate. The number of water molecules in the hydrate is not particularly limited, and examples of hydrates include those containing 1 to 7 water molecules. Preferred hydrates may include a dihydrate.

One embodiment of the tablet of the present invention contains zinc histidine hydrate as an active ingredient. Here, zinc histidine hydrate (JAN) is zinc histidine dihydrate.

The amount of active ingredients contained in the tablet can be selected appropriately so that it is the amount approved as a pharmaceutical (e.g., 25 mg or 50 mg equivalent to zinc). The content of the active ingredient may be, for example, 40% by mass to 75% by mass of the entire tablet. Alternatively, the content of the active ingredient may be 40% by mass or more, 45% by mass or more, 47% by mass or more, or 50% by mass or more, and 75% by mass or less, 70% by mass or less, 65% by mass or less, 60% by mass or less, 57% by mass or less, 55% by mass or less, or 52% by mass or less.

### MODE FOR CARRYING OUT THE INVENTION

In a preferred embodiment, the tablet of the present invention further contains one or more components selected from the group consisting of D-mannitol, sucrose, sorbitol, xylitol, corn starch, and potato starch, and crystalline cellulose.

The content of one or more components selected from the group consisting of D-mannitol, sucrose, sorbitol, xylitol, corn starch, and potato starch in the tablet is not particularly limited, but may be, for example, 5% by mass to 15% by mass of the entire tablet. Alternatively, the content of one or more components selected from the group consisting of D-mannitol, sucrose, sorbitol, xylitol, corn starch, and potato starch may be 5% by mass or more, 6% by mass or more, 7% by mass or more, 8% by mass, or 9% by mass or more, and 15% by mass or less, 14% by mass or less, 13% by mass or less, 12% by mass or less, or 11% by mass or less.

The content of crystalline cellulose in the tablet is not particularly limited, but may be, for example, 10% by mass to 30% by mass of the entire tablet. Alternatively, the content of crystalline cellulose may be 10% by mass or more, 15% by mass or more, 20% by mass or more, 22% by mass or more, or 24% by mass or more, and 30% by mass or less, 28% by mass or less, or 26% by mass or less.

In one embodiment, the content of zinc histidine hydrate in the tablet of the present invention is 40% by mass to 60% by mass of the entire tablet, and the content of one or more components selected from the group consisting of D-mannitol, sucrose, sorbitol, xylitol, corn starch, and potato starch is 5% by mass to 15% by mass of the entire tablet, and the content of crystalline cellulose is 20% by mass to 30% by mass of the entire tablet.

In one embodiment, the content of zinc histidine hydrate in the tablet of the present invention is 45% by mass to 55% by mass of the entire tablet, and the content of one or more components selected from the group consisting of D-mannitol, sucrose, sorbitol, xylitol, corn starch, and potato starch is 8% by mass to 12% by mass of the entire tablet, and the content of crystalline cellulose is 22% by mass to 28% by mass of the entire tablet.

The tablets of the present invention may be plain tablets or film-coated tablets. However, the contents (mass %) of each component in the tablets described above indicate the content ratios assuming that the tablets are plain tablets.

The tablets of the present invention do not contain lactose, fructose, or glucose. In particular, the tablets of the present invention do not contain lactose, fructose, or glucose as excipients. This is because the use of lactose, fructose, or glucose, especially as excipients, in the tablets may cause a decrease in solubility or discoloration during storage, thereby rendering the tablets incapable of maintaining the performance required for pharmaceuticals.

The tablets of the present invention may further contain, in addition to the above-mentioned components, additives commonly used in pharmaceuticals and active ingredients other than “zinc histidine or a solvate thereof.” One or two or more of these may be used. Examples of additives include, but are not limited to, excipients, disintegrants, lubricants, binders, coating agents, glossing agents, colorants, flavorings, sweeteners, and fragrances, and preferably excipients, disintegrants, binders, and lubricants.

Examples of the excipients include, but are not limited to, sugar alcohols such as erythritol, maltitol, and isomalt; amino acids such as glycine and alanine; silicates such as light anhydrous silica, synthetic aluminum silicate, magnesium aluminum metasilicate, and calcium silicate; talc; and titanium oxide.

In the present invention, one or more components selected from the group consisting of D-mannitol, sucrose, sorbitol, xylitol, corn starch, and potato starch, and crystalline cellulose may also be used as excipients, but the use of these components is not limited to excipients. That is, the present invention does not exclude the use of these components for purposes other than as fillers.

Examples of the disintegrant include, but are not limited to, crospovidone, calcium carmellose, carmellose, crosscarmellose, sodium crosscarmellose, and low-substituted hydroxypropyl cellulose. Preferred disintegrants include low-substituted hydroxypropyl cellulose.

Examples of lubricants include, but are not limited to, magnesium stearate, calcium stearate, talc, and sodium stearyl fumarate. Preferred lubricants include magnesium stearate.

Examples of binders may be selected from conventionally known materials and are not particularly limited, but examples include polyvinylpyrrolidone, hydroxypropyl cellulose, hypromellose, hypromellose phthalate, hydroxypropyl methylcellulose acetate succinate, ethyl cellulose, methyl cellulose, sodium alginate, gelatin, and carboxyvinyl polymers. Preferred binders include hydroxypropyl cellulose.

Examples of coating agents include, but are not limited to, water-soluble polymers, water-insoluble polymers, stomach-soluble polymers, enteric-soluble polymers, titanium oxide, polyethylene glycol, and talc.

Examples of water-soluble polymers include natural polymers such as sodium alginate; cellulose derivatives such as carmellose, carmellose sodium, carmellose calcium, hydroxypropyl cellulose, hypromellose, hydroxyethyl cellulose, hydroxymethyl cellulose, methyl cellulose, and carboxymethyl cellulose; water-soluble vinyl derivatives such as polyvinylpyrrolidone and polyvinyl alcohol.

Examples of water-insoluble polymers include ethyl cellulose, vinyl acetate polymer, aminoalkyl methacrylate copolymer, and ethyl acrylate/methyl methacrylate copolymer dispersion.

Examples of stomach-soluble polymers include aminoacetal compounds such as polyvinyl acetal diethylaminoacetate.

Examples of enteric-soluble polymers include enteric-soluble cellulose esters such as cellulose acetate propionate, hydroxypropyl methylcellulose acetate succinate, hypromellose phthalate, hydroxyethyl cellulose phthalate, carboxymethyl ethyl cellulose, and cellulose acetate phthalate.

Preferred coating agents include hydroxypropyl cellulose and hypromellose.

Examples of glossing agents include, but are not limited to, carnauba wax, hardened oil, vinyl acetate resin, salicylic acid wax, titanium oxide, stearic acid, calcium stearate, polyoxyethylene stearate 40, magnesium stearate, refined shellac, refined paraffin-carnauba wax mixture, cetanol, talc, white shellac, paraffin, polyvinylpyrrolidone (PVP), polyethylene glycol 1500, polyethylene glycol 4000, polyethylene glycol 6000, beeswax, glyceryl monostearate, and rosin.

Examples of coloring agents include food colorings, edible lake pigments, iron trioxide, and yellow iron trioxide.

Examples of flavor enhancers include citric acid, tartaric acid, malic acid, and ascorbic acid.

Examples of sweeteners include aspartame, acesulfame potassium, saccharin, sodium saccharin, dipotassium glycyrrhizinate, stevia, and somachin.

Examples of flavorings include anise oil, orange oil, chamomile oil, spearmint oil, cinnamon oil, clove oil, mint oil, bergamot oil, eucalyptus oil, lavender oil, lemon oil, rose oil, roman chamomile oil, and menthol.

It is preferable that the tablets of the present invention have a 15-minute dissolution value of 80% or more in the paddle method (test solution: water, rotate speed: 75 rpm). Furthermore, it is more preferable that the 15-minute dissolution value is 80% or more at the start of storage and after three months of storage under storage conditions without packaging, for example, at 40° C., 75% RH, in an open system. Such characteristics indicate that the tablets of the present invention are stable tablets that maintain the content of the active ingredient even after storage for a predetermined period.

The tablets of the present invention are preferably characterized by a lower rate of decrease (dissolution reduction rate) in the 15-minute dissolution value in the paddle method (test solution: water, rotate speed: 75 rpm) from the start of storage to three months after storage. In particular, it is preferable that the dissolution rate at the start of storage and after three months of storage under unpackaged storage conditions, for example, at 40° C. and 75% RH in an open system, is 80% or more, and that the dissolution reduction rate during this period is lower.

The dissolution reduction rate indicates the rate of decrease in the 15-minute dissolution value from the start of storage to three months after storage, and is a percentage value calculated using the following equation as the decrease in dissolution between the start of storage and three months after storage relative to the dissolution at the start of storage, i.e., the 15-minute dissolution value at the start of storage.

$$Dissolution reduction rate ( \% ) = [ ( Dissolution at start of storage ) - ( Dissolution aft er 3 months ) \text{]/} ( Dissolution at start of storage ) \times 100$$

The lower the dissolution reduction rate, the better the stability. Preferably, the dissolution reduction rate may be less than 15% or less than 10%, and more preferably less than 8%, and even more preferably less than 6%.

The tablets of the present invention can be manufactured by, for example, the following method.
Step 1: Granulating a mixture of the active ingredient (e.g., zinc histidine hydrate) and one or more ingredients selected from the group consisting of D-mannitol, sucrose, sorbitol, xylitol, corn starch, and potato starch, and crystalline cellulose to obtain a granulate (granulation step),
Step 2: Drying the obtained granules (drying step), and
Step 3: Tableting the dried granules to obtain plain tablets (tableting step).

Furthermore, in the case of producing coated tablets, the tablets may be manufactured by a method including the following Step 4 followed by Step 3.
Step 4: Film coating the plain tablets and then drying them (coating step).

These steps are usually performed in the order of Step 1 to Step 3 or Step 1 to Step 4, and may also include other steps. Examples of other steps include a milling step and a mixing step.
Step 1: Granulation process

Step 1 can be performed by a wet granulation method, such as a stirring granulation method or a fluidized bed granulation method, or by a dry granulation method. When zinc histidine hydrate is used as the active ingredient, for example, a mixture of zinc histidine hydrate and one or more components selected from the group consisting of D-mannitol, sucrose, sorbitol, xylitol, corn starch, and potato starch can be fed into a granulator and mixed, and then water or an aqueous solution of binders can be sprayed onto the mixture to obtain granules.

Following Step 1, the obtained granules can be put into a milling machine for milling. The milled granules can then be dried using a drying machine such as a fluidized bed dryer.
Step 2: Drying process

Step 2 is a process for producing granules suitable for tableting in Step 3 by removing the solvent (e.g., water) added in the granulation process.

Step 2 can be performed using a dryer such as a fluidized bed dryer. Specifically, the granules obtained in Step 1 are fed into the dryer and dried to the desired exhaust temperature while controlling the drying loss by the supply air temperature and exhaust temperature. If the drying loss exceeds the control range, the drying is repeated until the control range is reached. When the active ingredient is zinc histidine hydrate, setting the air supply temperature to, for example, 60° C. and drying until the exhaust temperature reaches 32° C. allows for the production of dried granules with a moisture content suitable for tableting while maintaining the hydration water of the zinc histidine hydrate raw material. Zinc histidine hydrate has a property of losing its hydration water (crystal water) at high temperatures and low humidity, resulting in dehydration. Therefore, if it is dried excessively (e.g., when the product temperature exceeds 40° C. for a certain period of time), it becomes difficult to maintain a moisture content suitable for tableting.
Step 3: Tableting process

Step 3 is a process of tableting the granules obtained in the previous step to obtain plain tablets. Tableting is performed by compression molding, and additives may be mixed at this time. The compression molding method is not particularly limited, and methods and equipment generally used for tablet molding, such as rotary tablet presses and single-shot tablet presses, may be used. The conditions for compression molding are not particularly limited as long as plain tablets can be obtained. In this step, tablets with the desired mass can be obtained.
Step 4: Coating process

Step 4 is a step of film coating the plain tablets and then drying them. The film coating method is not particularly limited, and methods generally used in the manufacture of film-coated tablets may be adopted. Film coating may be performed, for example, by wet spray coating the plain tablets with a suspension of a coating agent and a solvent using a coating machine. Examples of solvents include water or alcohols such as ethanol and isopropanol.

The drying following the film coating is a process for drying the solvent of the coating agent, and is not particularly limited as long as the loss of water in the active ingredient is suppressed, and conventional drying methods for coating agents in tablet manufacturing may be used.

The tablets obtained by coating may further be applied glossing agents by using conventional methods.

In one embodiment, the present invention provides a tablet obtained by:

a granulation step of granulating an active ingredient consisting of zinc histidine or a solvate thereof, D-mannitol, sucrose, sorbitol, xylitol, corn starch, and one or more components selected from potato starch, together with a binder;

a drying step of drying the obtained granules; and

a tableting step of tableting the dried granules, thereby obtaining a tablet containing zinc histidine or a solvate thereof as an active ingredient and having a 15-minute dissolution value in the paddle method (test solution: water, rotate speed: 75 rpm) of 80% or more. In this embodiment, the 15-minute dissolution value in the paddle method (test solution: water, rotate speed: 75 rpm) is preferably 80% or more at the start of measurement and after 3 months of storage at, for example, 40° C. and 75% RH in an open system.

### Examples

Hereinafter, the present invention is described with reference to specific embodiments, but the present invention is not limited to the embodiments, and it is understood that various changes and modifications thereof may be made by those skilled in the art without departing from the scope or intent of the invention as set forth in the appended claims.

### (Examples 1 and 2) Preparation of Test Samples 1 and 2

Zinc histidine hydrate, D-mannitol, and crystalline cellulose were mixed in a stirred granulator, hydroxypropyl cellulose and purified water were added, and the mixture was stirred and granulated. The granules were then granulated using a wet granulator. After that, the granules were dried using a fluidized bed dryer. Zinc histidine hydrate may lose its crystalline water due to over-drying, resulting in conversion to an anhydrous form. Therefore, the drying endpoint was evaluated based on the moisture loss of the granules. In all lots, the moisture loss values were slightly higher than the theoretical moisture content but remained at a constant level, and the water activity values were also low (0.3 or below), confirming that the material was sufficiently dried and no over-drying occurred. Next, the dried granules were milled, placed in a container rotary mixer, mixed with low substitution hydroxypropyl cellulose and magnesium stearate, and compressed into plain tablets (Test Sample 1). The total weight per plain tablet was 310 mg, the histidine zinc hydrate content was 156.7 mg (equivalent to 25 mg of zinc), the D-mannitol content was 33 mg, and the crystalline cellulose content was 80 mg.

Similarly, Test Sample 2 was prepared by changing the amounts of raw materials. Test Sample 2 had a total weight of 620 mg per plain tablet, a histidine zinc hydrate content of 313.4 mg (equivalent to 50 mg of zinc), a D-mannitol content of 66 mg, and a crystalline cellulose content of 160 mg.

### (Example 3) Preparation of Test Sample 3

Hypromellose and hydroxypropyl cellulose were dissolved in purified water, and a dispersion of titanium dioxide in purified water was mixed to prepare a coating solution. The plain tablets obtained in Example 1 (total weight per tablet was 310 mg, histidine zinc hydrate content was 157 mg (equivalent to 25 mg of zinc), D-mannitol content was 32.7 mg, and crystalline cellulose content was 80 mg) were sprayed with the coating solution, dried, and film-coated tablets (test sample 3) were obtained.

### (Examples 4, 5, 6, and 7) Preparation of Test Samples 6, 7, 8, and 9

Except for the use of sucrose (Test Sample 6), sorbitol (Test Sample 7), xylitol (Test Sample 8), and potato starch (Test Sample 9) in place of D-mannitol, the plain tablets were obtained in the same manner as in Test Sample 1. The total weight per plain tablet of the obtained test samples was 313.2 mg for test sample 6, 313.1 mg for test sample 7, 311.0 mg for test sample 8, and 310.5 mg for test sample 9. The content of zinc histidine hydrate in each test sample was 157 mg (equivalent to 25 mg of zinc), and the content of crystalline cellulose was 80 mg. Additionally, the content of sucrose (Test Sample 6), sorbitol (Test Sample 7), xylitol (Test Sample 8), and potato starch (Test Sample 9) in each test sample was 33 mg.

### (Comparative Example 1) Preparation of Test Sample 4

Except for the use of lactose hydrate instead of D-mannitol, plain tablets (Test Sample 4) were obtained in the same manner as in Test Sample 1.Test sample 4 had a total weight of 310 mg per plain tablet, a histidine zinc hydrate content of 157 mg (equivalent to 25 mg of zinc), a lactose hydrate content of 32.7 mg, and a crystalline cellulose content of 80 mg.

### (Comparative Example 2) Preparation of Test Sample 5

Using the coating solution used in the preparation of test sample 3, plain tablets (total weight per tablet: 314 mg, histidine zinc hydrate content: 157 mg (equivalent to 25 mg of zinc), lactose hydrate content: 35.2 mg, crystalline cellulose content: 81 mg) were prepared in the same manner as in Test Sample 4, and film-coated tablets (Test Sample 5) were obtained.

### (Comparative Examples 3 and 4) Preparation of Test Samples 10 and 11

Except for the use of fructose (Test Sample 10) and glucose (Test Sample 11) instead of D-mannitol, the plain tablets were obtained in the same manner as in Test Sample 1. The total weight per plain tablet of the obtained test samples was 313.1 mg for Test Sample 10 and 311.6 mg for Test Sample 11, and the content of zinc histidine hydrate in each test sample was 157 mg (equivalent to 25 mg of zinc) and the crystalline cellulose content was 80 mg. Additionally, the content of fructose (Test Sample 10) and glucose (Test Sample 11) in each test sample was 33 mg.

(Test Example 1) Solubility (50 rpm): Test Samples 1 to 3, 5

Test Samples were stored in glass bottles at 40° C. and 75% RH with the bottles open, and solubility was evaluated at the start of storage and after 3 months using the following method.

The dissolution rate (%) was measured at the start of storage and after three months using the paddle method (solvent: water, test liquid volume: 900 mL, rotate speed: 50 rpm) and the dissolution reduction rate was calculated using the following formula.

$$Active ingredient ( zinc ) dissolution reduction rate ( \% ) = [ ( dissolution rate at the start of storage ) - ( dissolution rate aft er three months ) \text{]/} ( dissolution rate at start of storage ) \times 100$$

The dissolution rate of Test Sample 1 (Example 1) at the start of storage was 83.9%, and after three months it was 82.5%, with a dissolution reduction rate of 1.67%.

The dissolution rate of Test Sample 2 (Example 2) at the start of storage was 86.9%, and after 3 months it was 81.8%, with a dissolution reduction rate of 5.87%.

The dissolution rates of Test Sample 3 (Example 3) at the start of storage and after three months were both 80% or higher and were almost identical, and no decrease in dissolution was observed.

The dissolution rate of Test Sample 5 (Comparative Example 2) at the start of storage was 88.8%, and after three months it was 75.4%, with a dissolution reduction rate of 15.1%.

(Test Example 2) Dissolution (rotate speed: 75 rpm): Test samples 1 and 2

Except for the rotate speed of 75 rpm, the dissolution reduction rate was measured in the same manner as in Test Example 1.

The dissolution rate of Test Sample 1 (Example 1) at the start of storage was 100%, and after three months, it was 98.2%, with a dissolution reduction rate of 1.8%.

The dissolution rates of Test Sample 2 (Example 2) at the start of storage and after three months were both 98.8%, and the dissolution reduction rate was 0%.

(Test example 2-1) Dissolution (rotation speed: 75 rpm): Test Samples 6 to 11

The dissolution reduction rate was measured in the same manner as in Test example 2.

The dissolution rates of Test Sample 6 (Example 4) at the start of storage and after three months were both 90% or higher and were almost identical, and no decrease in dissolution was observed.

The dissolution rate of Test Sample 7 (Example 5) at the start of storage was 94.1%, and after three months it was 91.5%, with a dissolution reduction rate of 2.6%.

The dissolution rate of Test Sample 8 (Example 6) at the start of storage was 96.0%, and after three months it was 91.0%, with a dissolution reduction rate of 5.0%.

The dissolution rates of Test Sample 9 (Example 7) at the start of storage and after three months were both 90% or higher and almost identical, and no decrease in dissolution was observed.

The dissolution rate of Test Sample 10 (Comparative Example 3) at the start of storage was 94.3%, and after three months, it was 37.7%, with a dissolution reduction rate of 56.6%.

The dissolution rate of Test Sample 11 (Comparative Example 4) at the start of storage was 91.1%, and after three months, it was 18.6%, with a dissolution reduction rate of 72.5%.

### (Test Example 3) Appearance: Test Samples 1 to 5

Test samples were stored in glass bottles at 40° C. and 75% RH with the bottles open, and their appearance was visually evaluated at the start of storage and after three months.

Test Samples 1 and 2 (Examples 1 and 2) were plain tablets, and there was no change in appearance between the start of storage and three months later.

Test Sample 3 (Example 3) was a white film-coated tablet, and there was no change in appearance between the start of storage and three months later.

Test Sample 4 (Comparative Example 1) was a white plain tablet at the start of storage, but changed to a light yellowish brown color after three months.

Test Sample 5 (Comparative Example 2) was a white film-coated tablet at the start of storage, but changed to a yellowish brown color after three months.

### (Test Example 3-1) Appearance: Test Samples 6 to 11

As in Test Example 3, appearance was evaluated visually at the start of storage and after three months.

Test Samples 6, 7, 8, and 9 (Examples 4, 5, 6, and 7) were white plain tablets, and there was no change in appearance between the start of storage and after three months. Test Samples 10 and 11 (Comparative Examples 3 and 4) were white plain tablets at the start of storage, but changed to light brown and brown, respectively, after three months.

### INDUSTRIAL APPLICABILITY

The present invention provides tablets containing zinc histidine or a solvate thereof as an active ingredient and having sufficient stability. The present invention also provides tablets containing zinc histidine or a solvate thereof as an active ingredient and having reduced dissolution and/or discoloration of the formulation.

1. A tablet comprising zinc histidine or a solvate thereof as an active ingredient, further comprising one or more ingredients selected from the group consisting of D-mannitol, sucrose, sorbitol, xylitol, corn starch, and potato starch; and crystalline cellulose; and excluding lactose, fructose, and glucose.

2. The tablet according to claim 1, wherein the content of the active ingredient is 40% by mass to 75% by mass of the entire tablet.

3. The tablet according to claim 1, wherein the content of one or more components selected from the group consisting of D-mannitol, sucrose, sorbitol, xylitol, corn starch, and potato starch is 5% by mass to 15% by mass of the entire tablet.

4. The tablet according to claim 1, wherein the content of crystalline cellulose is 10% by mass to 30% by mass of the entire tablet.

5. The tablet according to claim 1, wherein the active ingredient is contained in an amount equivalent to 25 mg or 50 mg of zinc.

6. The tablet according to claim 1, wherein the solvate is a hydrate.

7. The tablet according to claim 1, comprising zinc histidine hydrate as the active ingredient.

8. The tablet according to claim 1, having a 15-minute dissolution value in the paddle method (test solution: water, rotate speed: 75 rpm) is 80% or more.

9. The tablet according to claim 8, wherein the 15-minute dissolution value in the paddle method (test solution: water, rotate speed: 75 rpm) is 80% or more both at the start of storage and after 3 months under storage conditions of 40° C., 75% RH, and in an open system.

10. The tablet according to claim 9, wherein the 15-minute dissolution value in the paddle method (test solution: water, rotate speed: 75 rpm) decreases by less than 10% from the start of storage to three months under conditions of 40° C., 75% RH, and in an open system.

11. A tablet comprising zinc histidine or a solvate thereof as an active ingredient, having a 15-minute dissolution value of 80% or more in the paddle method (test solution: water, rotate speed: 75 rpm) (excluding lactose, fructose, and glucose), obtained by the following steps:
granulating zinc histidine or a solvate thereof, one or more components selected from the group consisting of D-mannitol, sucrose, sorbitol, xylitol, corn starch, and potato starch, and crystalline cellulose, with a binder; drying the obtained granulate; and tableting the dried granulate.

12. The tablet according to claim 11, wherein the 15-minute dissolution value in the paddle method (test solution: water, rotate speed: 75 rpm) is 80% or more at the start of storage and after 3 months of storage at 40° C., 75% RH, and in an open system.

13. The tablet according to claim 12, wherein the 15-minute dissolution value in the paddle method (test solution: water, rotate speed: 75 rpm) decreases by less than 10% from the start of storage to three months under conditions of 40° C., 75% RH, and in an open system.

14. The tablet according to claim 2, wherein the content of crystalline cellulose is 10% by mass to 30% by mass of the entire tablet.

15. The tablet according to claim 2, wherein the active ingredient is contained in an amount equivalent to 25 mg or 50 mg of zinc.

16. The tablet according to claim 2, wherein the solvate is a hydrate.

17. The tablet according to claim 3, wherein the content of crystalline cellulose is 10% by mass to 30% by mass of the entire tablet.

18. The tablet according to claim 3, wherein the active ingredient is contained in an amount equivalent to 25 mg or 50 mg of zinc.

19. The tablet according to claim 3, wherein the solvate is a hydrate.
